(19) United States
(12) Patent Application Publication
(10) Pub. No.: US 2023/0181667 A1
(43) Pub. Date: Jun. 15, 2023

(54) COMPOSITION FOR ENRICHING COSMETIC AND/OR DERMATOLOGICAL FOOD PRODUCTS

(71) Applicant: BLACK IDOL, Vron (FR)

(72) Inventors: Benoît LION, Viroflay (FR); Jérôme DE FRANCE, Nampont (FR)

(21) Appl. No.: 17/925,482

(22) PCT Filed: May 18, 2021
(86) PCT No.: PCT/FR2021/050875

§ 371 Date: Nov. 15, 2022

(30) Foreign Application Priority Data
May 18, 2020 (FR) 2004954

(51) Int. Cl. A61K 36/82; A61K 31/353; A61K 8/49; A61K 8/9789; A61Q 19/08; A61P 3/04; A61P 1/14; A61P 3/06; A23F 5/14; A23L 33/105; B65D 85/804

(52) U.S. Cl. CPC A61K 36/82; A61K 31/353; A61K 8/498; A61K 8/9789; A61Q 19/08; A61P 3/04; A61P 1/14; A61P 3/06; A23F 5/14; A23L 33/105; B65D 85/8067

(57) ABSTRACT

The present invention relates to a liquid composition for enriching aqueous or hydro-alcoholic food, cosmetic and/or dermatological products. The invention also relates to the use of said liquid composition and to the method for producing said liquid composition.

The present invention relates to a liquid composition for enriching food products, food supplements, cosmetic products, and/or dermatological products. The invention relates in particular to an aqueous or hydro-alcoholic liquid composition for enrichment with green tea extract. The invention also relates to the use of said liquid composition and to the method for producing said liquid composition.

### PRIOR ART

Green tea, in particular the leaves of the green tea plant Camellia sinensis, is the most consumed beverage in the world after water, and offers important health benefits. The compounds known to be responsible for these benefits are polyphenols and in particular catechins.

The main polyphenols of green tea (flavan-3-ol) are the catechins, i.e. (+)-catechin (C) and its stereoisomer and four derivatives, namely (−)-epicatechin (EC), (−)-epigallocatechin (EGC), (−)-epigallocatechin-3-gallate (EGCG), and (−)-epicatechin-3-gallate (ECg)

Although the amounts of catechins vary depending on factors that influence plant metabolism, such as light, temperature, nutrient availability, leaf age and genetic composition, they generally constitute 20-30% of the dry matter mass of fresh green tea in the total mass. Catechins account for about 80% of the total polyphenol content of green tea. Since they are rarely destroyed during the production process, catechins are an important part of commercial green tea extracts. Furthermore, catechins are less oxidized in green tea than in other teas (grey and black).

Green tea catechins have several health benefits that are often associated with their antioxidant activities, including scavenging of reactive oxygen and nitrogen species, chelation of heavy metals, and inhibition of oxidative enzymes such as lipoxygenase and cyclooxygenase. EGCG is the most interesting compound among the above-mentioned catechins because it has a strong antioxidant effect. In addition, EGCG has been shown to have an antimutagenic effect, an antibacterial effect and also a beneficial effect on blood cholesterol levels. The other catechins present in the tea are much less effective.

The effects of polyphenols, and in particular EGCG, on health depend on the quantity consumed but also and especially on their bioavailability. However, their bioavailability is low, which seems to be linked to a particular instability of these compounds in physiological conditions. In particular, EGCG is not very soluble and is difficult to dissolve in water, which makes it difficult to use for the supplementation of food products, such as drinks, or of cosmetic products, such as creams.

One possible strategy to increase the stability of green tea catechins and in particular EGCG is to protect them with antioxidants, thereby avoiding chemical changes caused by oxygen exposure. Patent document US2006057230 has proposed a method to increase the bioavailability of tea polyphenols by administering a tea polyphenol orally after an overnight fast prior to taking the tea polyphenol. However, there is no solution to improve the bioavailability of tea polyphenols, and in particular EGCG without a fasting period.

Indeed, G. Williamson et al, Int.J.Vitam. Nutri Res. 77 (3) 2007, 224-235 demonstrated that the addition of antioxidants (vitamins and carotenoids) had no effect on the bioavailability of polyphenols such as catechins.

Document EP 2008 524 discloses a method for producing a purified green tea extract containing non-polymeric catechins to improve the taste of the purified green tea extract by reducing the bitterness and astringency of said green tea.

The tea extract is treated there with the enzyme tannase, which, as known in the prior art at the date of this document, makes it possible to hydrolyze catechin esters, while precipitating, in order to eliminate astringent/bitter, fibrous and protein residues, with a mixture of alcohol and water and having a high alcohol proportion. The extract obtained is in powder form.

However, this document does not describe a green tea extract composition enriched with EGCG, and the enrichment of ground coffee with catechins cannot be implemented directly by adding such a dry powder of green tea extract, because the difference in particle size distribution between the ground coffee beans and the fine powder of said green tea extract would lead to problems of dispersion and distribution of said powder in the ground coffee beans. The preparation would then imperatively require a homogenization step of the two powder phases.

Thus, it is desirable that this enrichment with green tea extract, in particular with catechins, and particularly preferably with EGCG, can be carried out by a composition in liquid form to facilitate its addition and dispersion in the food product, food supplements, cosmetics or dermatological products, which complicates the technical problem, the EGCG being poorly soluble in water.

Lastly, in the particular case of ground coffee enrichment, the technical problem is further complicated by the fact that the moisture added during the coffee enrichment process by the liquid green tea extract can significantly promote the growth of fungi (mold), in particular due to the fact that the coffee is placed in a capsule, thus forming a sealed enclosure that prevents the elimination of water, thus developing aromas that are unpleasant to taste and potentially harmful to health.

Ground coffee capsules comprising green tea extracts are found on the market, for example “Fit Coffee.” These coffee capsules claiming detoxifying properties contain a mixture of natural ingredients, including green tea extract containing natural catechins.

However, this capsule does not claim properties related to catechins and their contents, which are not discussed in the product sheet, and does not seem to contain (−)-epigallocatechin-3-gallate (EGCG). Thus, the antioxidant properties of this product remain debatable and do not seem to be linked to catechins more than to the other products contained in the formulation, and there is still a need for a superior antioxidant contribution to the consumer.

The object of the present invention is therefore to solve the above-mentioned problems by providing a composition allowing the easy enrichment of food products, dietary supplements, but also cosmetic or dermatological products, in particular encapsulated ground coffee, with green tea extract, in particular with catechins, and in a particularly preferred manner with EGCG, with improved stability and bioavailability, and in amounts effective to obtain the desired effect, in particular on health.

It has now been found by the Applicant that a specific mixture of water, optionally with added ethanol, with a green tea extract comprising catechins, in particular EGCG, and a saturated or unsaturated dicarboxylic acid having a maximum of 6 carbons, preferably succinic acid, provides a liquid composition that solves these technical problems. In particular, the Applicant has demonstrated that the mixture of water, optionally with added ethanol, with a green tea extract forms a pasty-opaque mixture which turns into a transparent and much more fluid liquid once the saturated or unsaturated dicarboxylic acid having a maximum of 6 carbons, preferably succinic acid, is added.

This unexpected effect has, to a large extent, contributed to making the volumes of liquid added to the food product, food supplements, cosmetic products or dermatological products more accurate and reproducible. Another unexpected effect of this liquid composition is the very fast evaporation after contact with the ground coffee. A dry aggregate is observed after opening the doses of coffee thus made, without any trace of mold over time.

The object of the present invention is therefore to solve these technical problems by providing a liquid composition composed of an antioxidant-rich green tea extract containing catechins of the EGCG type and allowing said ground coffee capsules to be enriched, in order to address the aforementioned problems, on the one hand by making the green tea extract much more fluid by means of a saturated or unsaturated dicarboxylic acid having a maximum of 6 carbons, preferably succinic acid, in order to penetrate more rapidly into the ground coffee capsule, and on the other hand, by rapidly drying the solvents used so that they cannot catalyze the formation of mold before the sealing of said capsules.

Thus, a first object of the present invention relates to a liquid composition comprising or consisting of:
between 15 and 60%, preferably between 20 and 45%, based on the total weight of the composition, of green tea extract, said extract preferably comprising between 10 and 50%, preferably between 10 and 40%, more preferably between 15 and 35%, based on the total weight of the composition, of catechins, preferably comprising between 5 and 40%, preferably between 5 and 25%, more preferably between 9 and 20%, based on the total weight of the composition, of EGCG;
between 0.01 and 6%, preferably between 0.5 and 5.5%, preferably between 1 and 3%, based on the total weight of the composition, of a saturated or unsaturated dicarboxylic acid having a maximum of 6 carbons, preferably succinic acid;
between 0 and 75%, preferably between 17 and 62%, based on the total weight of the composition, of ethanol;
QSP water.

The term “liquid composition,” according to the present invention, shall be understood to mean a composition having a viscosity between 0.002 and 2.5 Pa.S, preferably between 0.004 and 0.1 Pa.S.

The production of the green tea extract used as a raw material is well known to a person skilled in the art. For example, green tea leaves are typically extracted with hot or cold water to form a solution containing tea catechins and caffeine. This green tea solution can be further concentrated to form either a concentrated extract solution or a dry powder. The extract solution or powder may contain stabilizers such as food approved acids, e.g. citric acid, ascorbic acid, isoascorbic acid and the like. Tea extract powders are also commercially available, for example from Guizhou Highyin Biological Product Co. in Guiyang, People's Republic of China, or Zhejang Zhongke Plant Technical Co. Ltd. in Hangzhou, Zhejang, People's Republic of China.

The term “catechins,” according to the present invention, shall be understood to mean in particular (−)-epicatechin (EC), (−)-epigallocatechin (EGC), (−)-epigallocatechin-3-gallate (EGCG) and (−)-epicatechin-3-gallate (ECg). It shall be understood according to the present invention that EGCG is epigallocatechin gallate, which is the ester of epigallocatechin and gallic acid. In particular, EGCG is considered to be the active ingredient of the liquid enrichment composition according to the present invention and will therefore be selected by a person skilled in the art in an amount effective for the desired effect, preferably between 9 and 20%, based on the total weight of the composition. This may correspond, for example for a coffee capsule of 4.5 g to 25 g to which 0.05 mL to 0.60 mL of liquid composition according to the present invention has been added, to the quantity of EGCG found in 1 g to 2 g of matcha tea.

The expression “saturated or unsaturated dicarboxylic acid having a maximum of 6 carbons” shall be understood within the meaning of the invention to mean organic compounds having two carboxyl functions with the molecular formula HOOC—R—COOH, where R can be an alkyl, alkenyl, alkynyl or aryl group. These compounds are easily soluble in water and comply with the standards imposed for use in the food industry. Indeed, these are authorized as food additives, and authorized for their cosmetic use. Said saturated or unsaturated dicarboxylic acid has a short carbon chain composed of a maximum of 6 carbons.

The term “saturated dicarboxylic acid” within the meaning of the invention shall be understood to mean a linear saturated dicarboxylic acid of general formula HO₂C(CH₂)ₙCO₂H.

The term “saturated” means that said dicarboxylic acid has a total number of atoms equal to that which can be deduced from the maximum valence of each of its constituent atoms considered individually (i.e., there is no double bond, triple bond or ring).

Preferably, said saturated dicarboxylic acid is succinic acid.

The term “unsaturated dicarboxylic acid” shall be understood to mean a dicarboxylic acid having a total number of atoms lower than that which can be deduced from the maximum valence of each of its constituent atoms considered individually. It may thus include double or triple bonds.

Preferably, said unsaturated dicarboxylic acid is selected from maleic acid, fumaric acid, cis-glutaconic acid, or trans-glutaconic acid.

Preferably, said saturated or unsaturated dicarboxylic acid having a maximum of 6 carbons is selected from maleic acid, fumaric acid, succinic acid, cis-glutaconic acid, or trans-glutaconic acid, preferably succinic acid.

The expression “having a maximum of 6 carbons” shall be understood to mean that the dicarboxylic acid in the sense of the invention has a number of carbons between 2 and 6.

“Succinic acid” shall be understood to mean an aliphatic dicarboxylic acid, also known as 1,4-dioic acid. Succinic acid as a food additive is listed as an acidity regulator in the Codex alimentarius, and as a flavor enhancer of natural origin. It is listed under the code E363 and is permitted in an amount of between 3 and 6 grams per kilo in the European Union in fermented dairy products, soups, beverage mixes and desserts. Apart from processed food, it is also found in cosmetics, especially as an odor or taste inhibitor, and in pharmaceutical products.

The saturated or unsaturated dicarboxylic acid having a maximum of 6 carbons, preferably succinic acid, makes it possible to reduce the density and viscosity of the liquid composition composed of green tea extract according to the invention, which in its absence is particularly dense and viscous, being very heavily charged with said green tea extract. However, like any very dense liquid, its penetration into the mass of ground coffee already distributed in the capsule is too slow to be compatible with the rate of sealing of the capsules. The absence of saturated or unsaturated dicarboxylic acid having a maximum of 6 carbons, preferably succinic acid, would result in high losses due to the poor sealing of said ground coffee capsules.

Preferably, the liquid composition according to the invention is characterized in that the ethanol is absolute ethanol, or 95% or 96% ethanol.

The liquid composition according to the present invention further comprises at least one excipient. A person skilled in the art will choose one or more acceptable excipients depending on the product to be ultimately enriched and its use. Thus, for a product to be enriched for food use, they will choose excipients which are acceptable to the control authorities (FDA in the United States, EFSA in Europe).

Preferably, the liquid composition according to the invention is characterized in that the green tea extract comprises between 10 and 40%, preferably between 15 and 35%, based on the total weight of the composition, of catechins.

According to one embodiment, the ground coffee liquid enrichment composition comprises between 34% and 44%, based on the total weight of the composition, of catechins.

According to one embodiment, the ground coffee liquid enrichment composition comprises between 27% and 35%, based on the total weight of the composition, of catechins.

According to one embodiment, the ground coffee liquid enrichment composition comprises between 37% and 48%, based on the total weight of the composition, of catechins.

Preferably, the liquid composition according to the invention is characterized in that the green tea extract comprises between 5 and 25%, preferably between 9 and 20%, based on the total weight of the composition, of EGCG.

According to one embodiment, the ground coffee liquid enrichment composition comprises between 17% and 31%, based on the total weight of the composition, of EGCG.

According to one embodiment, the ground coffee liquid enrichment composition comprises between 14% and 26%, based on the total weight of the composition, of EGCG.

According to one embodiment, the ground coffee liquid enrichment composition comprises between 19% and 35%, based on the total weight of the composition, of EGCG.

Preferably, the liquid composition according to the invention is characterized in that it is a hydro-alcoholic solution or suspension

According to a second object, the present invention relates to the use of the composition according to the present invention for the enrichment with green tea extract, preferably the enrichment with catechins, particularly preferably the enrichment with EGCG, of food products intended for human beings or animals, preferably ground coffee, of food supplements, of cosmetic products, and/or of dermatological products.

Green tea extract, preferably the catechins it contains, particularly preferably EGCG, presented in the form of a liquid enrichment composition as described above has a high antioxidant activity and can therefore be used as an antioxidant and/or anti-aging agent, for various food products, animal feeds, dietary supplements, cosmetic products, or dermatological products.

Furthermore, green tea extract, preferably the catechins contained therein, particularly preferably EGCG, has an antimutagenic effect, an antibacterial effect and also a beneficial effect on the blood cholesterol level. Thus, the liquid enrichment composition according to the present invention is useful in preventive or health care preparations, as a pharmaceutical or parapharmaceutical, preferably as a food supplement or enriched food product, in particular for the prevention of cardiovascular pathologies, as an anti-cholesterol, for cancer prevention, prevention of skin infections, weight reduction, or increase of muscle development.

Green tea extract, preferably the catechins contained therein, particularly preferably EGCG, also has an anxiolytic effect. Thus, the liquid enrichment composition according to the present invention is useful in preventive or health care preparations, as a pharmaceutical or parapharmaceutical, preferably as a food supplement or enriched food product, in particular for the prevention or reduction of anxiety, as an anti-stress agent, relaxation agent, for the prevention or improvement of sleep disorders, or as a sleep-inducing agent.

Furthermore, green tea extract, preferably the catechins contained therein, particularly preferably EGCG, has effects on endothelial functions. Thus, the liquid enrichment composition according to the present invention is useful in preventive or health care preparations, as a pharmaceutical or parapharmaceutical, preferably as a food supplement or enriched food product, in particular for maintaining cognitive functions and memory, increasing concentration and mental faculties, or increasing lucidity, energy and vivacity.

A person skilled in the art will adjust the quantity of liquid composition to be added to the food, cosmetic or dermatological product or food supplement on the basis of their general knowledge so as to achieve the desired benefit. Thus, according to one embodiment, between 0.1 and 40 ml of said liquid composition can be added per kg of food, cosmetic or dermatological product or food supplement for an antioxidant effect. A person skilled in the art will know how to adapt the quantity of liquid composition to be added to the product in order to obtain an effective concentration of catechins and in particular of EGCG, according to the desired use and the benefit to be achieved, and which does not exceed the daily recommendations of the control authorities (in particular the FDA and EFSA).

According to an embodiment of the invention, the food product is a beverage. According to an embodiment of the invention, the food product is a powder. In particular, the food product may be a powder (chocolate powder, milk powder, chicory, meal drink, etc.) ready to use for reconstituting a beverage after being added to a liquid (water, milk) and after having dissolved.

Preferably, the use of the composition according to the present invention is for the enrichment with EGCG of ground coffee comprised in a single-use coffee capsule, of the Nespresso® compatible type or of the dosed pod type. These capsules traditionally contain between 5 and 6 g of ground coffee for preparation of an espresso, and up to 25 g for preparation of a long coffee or double espresso.

As an example, 0.16 ml of the liquid composition according to the invention, dosed between 9 and 20% with EGCG, can be added to 5 to 6 g of ground coffee in an espresso type capsule, and 0.48 ml of the liquid composition according to the invention, dosed between 9 and 20% with EGCG, can be added to 15 to 20 g of ground coffee in a long coffee type capsule.

According to another example, 0.26 ml of the liquid composition according to the invention, dosed between 9 and 20% with EGCG, can be added to 4.5 to 6 g of ground coffee in an espresso type capsule, and 0.60 ml of the liquid composition according to the invention, dosed between 9 and 20% with EGCG, can be added to 20 to 25 g of ground coffee in a capsule provided for this purpose.

These single-use ground coffee capsules are used to make a cup of coffee through the combined use of a coffee machine, preferably under high pressure, and a capsule.

The capsules are for example made of aluminum foil and covered with an internal food film that prevents any contact between the aluminum and the coffee. Alternatively, the capsules are made of plastic or a biodegradable and compostable composite material. When the capsule is inserted into the machine, the top is perforated. Then, once the machine is activated, water is pumped hot and under high pressure into the capsule.

Preferably, according to the present invention, each capsule comprising between 4.5 and 25 grams of ground coffee is enriched with 0.05 ml to 0.60 milliliters of said composition according to the invention.

According to a preferred embodiment, each capsule comprising 4.5 to 25 grams of ground coffee is enriched with 0.25 ml to 0.60 milliliters of said composition, preferably 0.26 ml.

According to another preferred embodiment, each capsule comprising 5 to 6 grams of ground coffee is enriched with 0.05 ml to 0.25 milliliters of said composition, preferably between 0.14 and 0.18 ml, particularly preferably 0.16 ml.

According to another preferred embodiment, the use of the composition according to the present invention is characterized in that each capsule comprising 4.5 to 25 grams of ground coffee is enriched with 0.15 ml to 0.60 milliliters of said composition, preferably 0.26 ml.

According to a third object, the present invention relates to a method for enriching ground coffee in a single-use coffee capsule comprising, in particular, a step of adding the composition according to the present invention to said ground coffee before sealing the capsule.

The liquid composition according to the present invention is preferably added by means of a liquid distribution system. This system is capable of delivering precise volumes of liquid by means of an ad-hoc pump generating a constant flow of the liquid, the temperature of which needs to be regulated to optimize its liquidity.

According to a fourth object, the present invention relates to a single-use coffee capsule enriched with green tea extract, preferably with catechins, more preferably with EGCG, obtained by the method according to the present invention.

According to a fifth object, the present invention relates to a method for producing the composition according to the invention, characterized in that it comprises the following steps:
a. producing a solvent system by mixing said ethanol, if present in the composition, and said water
b. adding to the solvent system thus obtained in step a) said green tea extract and mixing
c. adding to the mixture obtained in step b) said saturated or unsaturated dicarboxylic acid having a maximum of 6 carbons, preferably succinic acid, and mixing to obtain a liquid composition.

As a non-limiting example, we will now describe an embodiment of the invention

### EXAMPLES

### Example 1

### Examples of Formulations of Single-Use Coffee Capsules Enriched with Green Tea Extract Containing Catechins, including EGCG

A. Ground Coffee Liquid Enrichment Composition for a Slimming Effect

TABLE 1

| | quantity (mg) | % by weight |
|---|---|---|
| green tea extract | 91 | 44.3 |
| of which catechin | 68.25 | 33.2 |
| of which EGCG | 40 | 19.5 |
| succinic acid (E363) | 5 | 2.4 |
| water | 74.3 | 36.2 |
| ethanol 95 or 95% | 35 | 17.1 |
| total volume | 0.16 ml | |
| total | 205.3 | 100 |

B. Ground Coffee Liquid Enrichment Composition for a Digestion-Promoting Effect

TABLE 2

| | quantity (mg) | % by weight |
|---|---|---|
| green tea extract | 45.5 | 20.8 |
| of which catechin | 34.1 | 15.6 |
| of which EGCG | 20 | 9.1 |
| succinic acid (E363) | 2.5 | 1.1 |
| water | 37.1 | 16.9 |
| ethanol 95 or 95% | 134 | 61.2 |
| total volume | 0.16 ml | |
| total | 219.1 | 100 |

C. Ground Coffee Liquid Enrichment Composition for an Anti-Cholesterol Effect

TABLE 3

| | quantity (mg) | % by weight |
|---|---|---|
| green tea extract | 68.25 | 32.2 |
| of which catechin | 51.19 | 24.1 |
| of which EGCG | 30 | 14.1 |
| succinic acid (E363) | 3.7 | 1.7 |
| water | 55.7 | 26.3 |
| ethanol 95 or 95% | 84.4 | 39.8 |
| total volume | 0.16 ml | |
| total | 212.05 | 100 |

D. Ground Coffee Liquid Enrichment Composition for an Anti-Aging Effect

TABLE 4

| | quantity (mg) | % by weight |
|---|---|---|
| green tea extract | 68.25 | 32.2 |
| of which catechin | 51.19 | 24.1 |
| of which EGCG | 30 | 14.1 |
| succinic acid (E363) | 3.7 | 1.7 |
| water | 55.7 | 26.3 |
| ethanol 95 or 95% | 84.4 | 39.8 |
| total volume | 0.16 ml | |
| total | 212.05 | 100 |

2) Enrichment of the coffee capsule

Each volume of 0.16 ml solution (Tables 1, 2, 3 and 4) is added to a Nespresso® type capsule for preparation of an espresso, on the freshly ground coffee. It is noted that the liquid evaporates within a few seconds and that a dry coffee aggregate forms at the point where the liquid composition was added. The capsule is sealed and ready to use.
3) Obtaining a cup of enriched coffee with benefits for the consumer

The capsule thus obtained is inserted into a Nespresso® type machine, adding hot water under pressure, to make a cup of coffee enriched with green tea extract, catechins and EGCG, with the stated benefits (slimming, facilitating digestion, anti-cholesterol, anti-aging) being achieved for the consumer.

### Example 2

### Examples of Formulations of Single-Use Coffee Capsules Enriched with Green Tea Extract Containing Catechins, including EGCG

A. Ground Coffee Liquid Enrichment Composition for a Slimming Effect

The percentages by weight in Tables 5 to 8 are expressed for the entire composition obtained in the capsule (coffee+composition according to the invention).

TABLE 5

| | | % by weight |
|---|---|---|
| | quantity (mg) | (rounded to 0.01) |
| green tea extract | 139.1 | 2.64 |
| of which catechin | 107 | 2.02 |
| of which EGCG | 58 | 1.10 |
| succinic acid (E363) | 1.3 | 0.03 |
| water | 90 | 1.70 |
| ethanol 95 or 95% | 57.0 | 1.10 |
| total volume | 0.26 ml | |
| capsule of 5 g | 5000 | 94.60 |
| total | 5287 | 100 |

B. Ground Coffee Liquid Enrichment Composition for a Digestion-Promoting Effect

TABLE 6

| | | % by weight |
|---|---|---|
| | quantity (mg) | (rounded to 0.01) |
| green tea extract | 106 | 2.01 |
| of which catechin | 82 | 1.56 |
| of which EGCG | 44 | 0.83 |
| succinic acid (E363) | 1 | 0.02 |
| water | 80 | 1.52 |
| ethanol 95 or 95% | 83 | 1.57 |
| total volume | 0.26 ml | |
| capsule of 5 g | 5000 | 94.90 |
| total | 5270 | 100 |

C. Ground Coffee Liquid Enrichment Composition for an Anti-Cholesterol Effect

TABLE 7

| | | % by weight |
|---|---|---|
| | quantity (mg) | (rounded to 0.01) |
| green tea extract | 139.1 | 2.63 |
| of which catechin | 107 | 2.02 |
| of which EGCG | 58 | 1.10 |
| succinic acid (E363) | 1.3 | 0.02 |
| water | 90 | 1.70 |
| ethanol 95 or 95% | 57.0 | 1.10 |
| total volume | 0.26 ml | |
| capsule of 5 g | 5000 | 94.60 |
| total | 5287 | 100 |

D. Ground Coffee Liquid Enrichment Composition for an Anti-Aging Effect

TABLE 8

| | | % by weight |
|---|---|---|
| | quantity (mg) | (rounded to 0.01) |
| green tea extract | 159 | 3.00 |
| of which catechin | 122 | 2.30 |
| of which EGCG | 66 | 1.25 |
| succinic acid (E363) | 1.5 | 0.03 |
| water | 100 | 1.90 |
| ethanol 95 or 95% | 38 | 0.72 |
| total volume | 0.26 ml | |
| capsule of 5 g | 5000 | 94.36 |
| total | 5299 | 100 |

2) Enrichment of the coffee capsule

Each volume of solution of 0.26 ml (Tables 5, 6, 7 and 8) is added in a 5 g Nespresso® type capsule for the preparation of an espresso type coffee, on the freshly ground coffee. It is noted that the liquid evaporates within a few seconds, and that a dry coffee aggregate forms at the point where the liquid composition was added. The capsule is sealed and ready to use.
3) Obtaining a cup of enriched coffee with benefits for the consumer

The capsule thus obtained is inserted into a Nespresso® type machine, adding hot water under pressure, to make a cup of coffee enriched with green tea extract, catechins and EGCG, with the stated benefits (slimming, facilitating digestion, anti-cholesterol, anti-aging) being achieved for the consumer.

### Example 3

### Examples of Formulations of Single-Use Coffee Capsules Enriched with Green Tea Extract Containing Catechins, including EGCG

A. Ground Coffee Liquid Enrichment Composition for a Slimming Effect

The percentages by weight in Tables 9 to 12 are expressed for the entire composition obtained in the capsule (coffee+composition according to the invention).

TABLE 9

| | | % by weight |
|---|---|---|
| | quantity (mg) | (rounded to 0.01) |
| green tea extract | 139.1 | 0.55 |
| of which catechin | 107 | 0.42 |
| of which EGCG | 58 | 0.3 |
| succinic acid (E363) | 1.3 | 0.01 |
| water | 90 | 0.36 |
| ethanol 95 or 95% | 57.0 | 0.23 |
| total volume | 0.26 ml | |
| capsule of 25 g | 25000 | 98.86 |
| total | 5287 | 100 |

B. Ground Coffee Liquid Enrichment Composition for a Digestion-Promoting Effect

TABLE 10

| | | % by weight |
|---|---|---|
| | quantity (mg) | (rounded to 0.0001) |
| green tea extract | 106 | 0.4195 |
| of which catechin | 82 | 0.3245 |
| of which EGCG | 44 | 0.1741 |
| succinic acid (E363) | 1 | 0.0004 |
| water | 80 | 0.3166 |
| ethanol 95 or 95% | 83 | 0.3285 |
| total volume | 0.26 ml | |
| capsule of 25 g | 25000 | 98.9315 |
| total | 25270 | 100 |

C. Ground Coffee Liquid Enrichment Composition for an Anti-Cholesterol Effect

TABLE 11

| | | % by weight |
|---|---|---|
| | quantity (mg) | (rounded to 0.01) |
| green tea extract | 139.1 | 0.55 |
| of which catechin | 107 | 0.42 |
| of which EGCG | 58 | 0.23 |
| succinic acid (E363) | 1.3 | 0.01 |
| water | 90 | 0.36 |
| ethanol 95 or 95% | 57.0 | 0.23 |
| total volume | 0.26 ml | |
| capsule of 25 g | 25000 | 98.86 |
| total | 25287 | 100 |

D. Ground Coffee Liquid Enrichment Composition for an Anti-Aging Effect

TABLE 12

| | | % by weight |
|---|---|---|
| | quantity (mg) | (rounded to 0.01) |
| green tea extract | 159 | 0.63 |
| of which catechin | 122 | 0.48 |
| of which EGCG | 66 | 0.26 |
| succinic acid (E363) | 1.5 | 0.01 |
| water | 100 | 0.40 |
| ethanol 95 or 95% | 38 | 0.15 |
| total volume | 0.26 ml | |
| capsule of 25 g | 25000 | 98.82 |
| total | 25299 | 100 |

2) Enrichment of the coffee capsule

Each volume of solution of 0.26 ml (Tables 9, 10, 11 and 12) is added in a capsule of 25 g for the preparation of a long coffee, on the freshly ground coffee. It is noted that the liquid evaporates within a few seconds and that a dry coffee aggregate is formed where the liquid composition was added. The capsule is sealed and ready to use.
3) Obtaining a cup of enriched coffee with benefits for the consumer

The capsule thus obtained is inserted into a Nespresso® type machine, adding hot water under pressure, to make a cup of coffee enriched with green tea extract, catechins and EGCG, with the stated benefits (slimming, facilitating digestion, anti-cholesterol, anti-aging) being achieved for the consumer.

1. A liquid composition comprising:
between 15 and 60%, preferably between 20 and 45%, based on the total weight of the composition, of green tea extract, said extract comprising between 10 and 50%, preferably between 10 and 40%, more preferably between 15 and 35%, based on the total weight of the composition, of catechins, comprising between 5 and 40%, preferably between 5 and 25%, more preferably between 9 and 20%, based on the total weight of the composition, of EGCG;
between 0.01 and 6%, preferably between 0.5 and 5.5%, preferably between 1 and 3%, based on the total weight of the composition, of a saturated or unsaturated dicarboxylic acid having a maximum of 6 carbons;
between 0 and 75%, preferably between 17 and 62%, based on the total weight of the composition, of ethanol;
QSP water.

2. The liquid composition according to claim 1, characterized in that the green tea extract comprises between 10 and 40%, preferably between 15 and 35%, based on the total weight of the composition, of catechins.

3. The liquid composition according to claim 1, characterized in that the green tea extract comprises between 5 and 25%, preferably between 9 and 20%, based on the total weight of the composition, of EGCG.

4. The liquid composition according to claim 1, characterized in that said saturated or unsaturated dicarboxylic acid having a maximum of 6 carbons is selected from maleic acid, fumaric acid, succinic acid, cis-glutaconic acid, or trans-glutaconic acid.

5. The liquid composition according to claim 4, characterized in that said saturated or unsaturated dicarboxylic acid having a maximum of 6 carbons is succinic acid.

6. The liquid composition according to claim 1, characterized in that it is a hydro-alcoholic solution or suspension.

7. The liquid composition according to claim 1, characterized in that the ethanol is absolute ethanol, or 95% or 96% ethanol.

8. Use of the composition according to claim 1 for the enrichment with green tea extract, preferably the enrichment with catechin, particularly preferably the enrichment with EGCG, of food products intended for human beings or animals, preferably ground coffee, of food supplements for human beings or animals, of cosmetic products, and/or of dermatological products.

9. The use according to claim 8 of said composition for the enrichment with EGCG of ground coffee comprised in a single-use coffee capsule.

10. The use according to claim 9, characterized in that each capsule comprising 4.5 to 25 grams of ground coffee is enriched with 0.05 ml to 0.60 milliliters of said composition.

11. The use according to claim 10, characterized in that each capsule comprising 4.5 to 25 grams of ground coffee is enriched with 0.15 ml to 0.60 milliliters of said composition, preferably 0.26 ml.

12. The use according to claim 10, characterized in that each capsule comprising 5 to 6 grams of ground coffee is enriched with 0.05 ml to 0.25 milliliters of said composition, preferably 0.16 ml.

13. The use according to claim 10, characterized in that each capsule comprising 4.5 to 25 grams of ground coffee is enriched with 0.25 ml to 0.60 milliliters of said composition, preferably 0.26 ml.

14. A method for enriching ground coffee in a single-use coffee capsule comprising in particular a step of adding the composition according to claim 1 to said ground coffee before sealing the capsule.

15. A single-use coffee capsule enriched with green tea extract, preferably with catechins, more preferably with EGCG, obtained by the method according to claim 14.

16. A method for producing the composition according to claim 1, characterized in that it comprises the following steps:
a. producing a solvent system by mixing said ethanol, if present in the composition, and said water
b. adding to the solvent system thus obtained in step a) said green tea extract and mixing
c. adding to the mixture obtained in step b) said saturated or unsaturated dicarboxylic acid having a maximum of 6 carbons, preferably succinic acid, and mixing to obtain a liquid composition.
